(19) United States
(12) Patent Application Publication
(10) Pub. No.: US 2005/0277677 A1
(43) Pub. Date: Dec. 15, 2005

(54) Method for treating emesis with ghrelin agonists

(76) Inventors: Mark Louis Heiman, Indianapolis, IN (US); Dana Kevin Sindelar, Indianapolis, IN (US)

Correspondence Address: ELI LILLY AND COMPANY;PATENT DIVISION, P.O. BOX 6288, INDIANAPOLIS, IN, 46206-6288, US

(21) Appl. No.: 11/130,766

(22) Filed: May 17, 2005

Related U.S. Application Data
(60) Provisional application No. 60/578,771, filed on Jun. 10, 2004.

(51) Int. Cl. A61K 31/454; A61K 31/4178; A61K 31/4172

(52) U.S. Cl. 514/326; 514/397; 514/400

(57) ABSTRACT

The present invention relates to a method comprising administering to a patient diagnosed as being in need of treatment for nausea, emesis, or symptoms associated therewith comprising administering to a patient in need thereof a compound of formula (I) 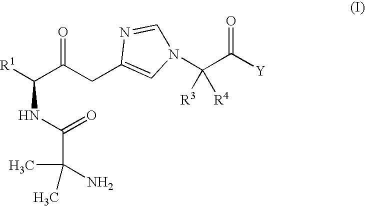 wherein:

R¹ is C₆H₅CH₂OCH₂—, C₆H₅(CH₂)₃— or indol-3-ylmethyl;

Y is pyrrolidinyl, 4-methyl-piperidinyl or NR²R²;

R² are each independently C₁-C₆ alkyl;

R³ is 2-napthyl or phenyl para-substituted by W;

W is H, F, CF₃, C₁-C₆ alkoxy or phenyl; and

R⁴ is H or CH₃; or a pharmaceutically acceptable salt or solvate thereof, in an amount that is effective in treating nausea, emesis, or symptoms associated therewith in said patient.

### BACKGROUND OF THE INVENTION

Nausea and vomiting can follow the administration of many drugs, particularly anticancer or chemotherapeutic agents. The symptoms also often accompany infectious and non-infectious gastrointestinal disorders.

The initial manifestations of the vomiting response often involves nausea, in which gastric tone is reduced, gastric peristalsis is reduced or absent and the tone of the duodenum and upper jejunum is increased, such that their contents reflux. Ultimately, the upper portion of the stomach relaxes while the pylorus constricts, and the coordinated contraction of the diaphragm and abdominal muscles leads to expulsion of gastric contents. Goodman and Gilman's, The Pharmacological Basis of Therapeutics, 8th Edition, Pergamon Press, New York, pp. 925-928 (1990).

Many workers have studied the effects of various drugs in alleviating the symptoms of emesis. In the Goodman and Gilman text, the authors mention metoclopramide (MTC), a benzamide, as a dopaminergic antagonist with important antiemetic uses. Benzodiazepines, another class of drugs, can enhance the effectiveness of antiemetic regimens and are thought to be beneficial in the prevention of anticipatory emesis. Also, dexamethasone (DEX) and other glucocorticoids are said to have antiemetic effects and may improve the efficacy of antiemetic regimens in some cancer patients. The authors name six phenothiazine compounds, one butyrophenone, two benzamides including metoclopramide and two cannabinoids as agents used in the treatment of nausea.

Goodman and Gilman describe metoclopramide as being well tolerated in high intravenous dosages and being widely used to control emesis during cancer chemotherapy, especially when highly emetogenic agents, such as cisplatin or cyclophosphamide, are used. Metoclopramide has been combined with diphenhydramine (DPH). Regimens that are reportedly effective in countering vomiting induced by cisplatin or cyclophosphamide include those that utilize the intravenous administration of metoclopramide and dexamethasone in combination with lorazepam plus benztropine or droperidol plus diphenhydramine.

In an article by Markman et al., in the New England Journal of Medicine, Vol. 311, pp. 549-552 (1984), the authors compare the antiemetic effects of dexamethasone with prochlorperazine. It is concluded that there is less nausea and vomiting with dexamethasone than with the prochlorperazine. The authors also refer to two studies comparing the efficacy of high-dose dexamethasone and high-dose metoclopramide. The dexamethasone was said to be more effective than metoclopramide in controlling chemotherapy-induced nausea and vomiting and was preferred by the patients treated.

In a review of metoclopramide, in Drugs 25:451-494 (1983), at page 453, the authors assert that controlled trials have shown oral metoclopramide (30-40 mg daily) alleviates the symptoms of gastro-oesophageal reflux relative to placebo and increases lower oesophageal sphincter pressure.

In another publication, Roila, in Oncology 50:163-167 (1993), discusses the results of administering ondansetron plus dexamethasone, compared to the standard metoclopramide combination. In the paper, a composition comprising metoclopramide (3 mg/kg), dexamethasone (20 mg) and diphenhydramine (50 mg), administered intravenously, is compared with a composition of ondansetron (0.15 mg/kg) and dexamethasone (20 mg), administered intravenously. The results, summarized in the last line of the abstract at page 163, advises that ondansetron plus dexamethasone is a more effective and better tolerated antiemetic regimen compared with metoclopramide plus dexamethasone and diphenhydramine for the prevention of acute cisplatin-induced emesis.

In the patented literature, such as U.S. Pat. No. 5,039,528, metoclopramide is described as a suitable agent for suppressing emesis associated with cancer therapy. However, the patentee notes, this agent exhibits effective antiemetic activity only when used at high doses. In U.S. Pat. No. 5,482,716, the patentees indicate that studies show the antiemetic properties of carbazolone(1,2,3,9-tetrahydro-9-methyl-3-[(2-methyl-1H-imidazol-1-yl)methyl]-4H-carbazol-4-one) are enhanced by administering the compound in conjunction with dexamethasone, a systemic anti-inflammatory corticosteroid that is known to have antiemetic properties. In U.S. Pat. No.5,310,561, in Example 6, ondansetron is used with metoclopramide, haloperidol or droperidol, and dexamethasone, among others.

### BRIEF SUMMARY OF THE INVENTION

The present invention relates to a method comprising administering to a patient in need of treatment for nausea, emesis, or symptoms associated therewith a compound of formula (I) 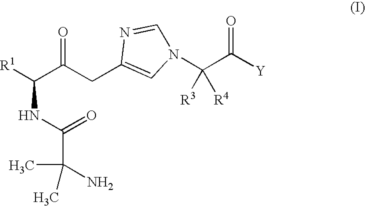
wherein:

R¹ is C₆H₅CH₂OCH₂—, C₆H₅(CH₂)₃— or indol-3-ylmethyl;

Y is pyrrolidinyl, 4-methyl-piperidinyl or NR²R²;

R² are each independently C₁ -C₆ alkyl;

R³ is 2-napthyl or phenyl para-substituted by W;

W is H, F, CF₃, C₁-C₆ alkoxy or phenyl; and

R⁴ is H or CH₃;

or a pharmaceutically acceptable salt or solvate thereof, in an amount that is effective in treating nausea, emesis, or symptoms associated therewith in said patient.

### DETAILED DESCRIPTION OF THE INVENTION

General terms used in the description of compounds herein described bear their usual meanings. For example, the term “C₁-C₆ alkyl” refers to straight or branched, monovalent, saturated aliphatic chains of 1 to 6 carbon atoms and includes, but is not limited to, methyl, ethyl, propyl, isopropyl, butyl, isobutyl, sec-butyl, tert-butyl, pentyl, isopentyl, hexyl, and the like. The term “C₁-C₆ alkyl” includes within its definition the term “C₁-C₄ alkyl”.

The term “C₁-C₆ alkoxy” represents a straight or branched alkyl chain having from one to six carbon atoms attached to an oxygen atom. Typical “C₁-C₆ alkoxy” groups include methoxy, ethoxy, propoxy, isopropoxy, butoxy, tert-butoxy, pentoxy, and the like. The term “C₁-C₆ alkoxy” includes within its definition the term “C₁-C₄ alkoxy”.

The compounds used in the method of the present invention have two chiral centers. As a consequence of these chiral centers, the compounds of the present invention occur as diastereomers and mixtures of diastereomers. All asymmetric forms, individual isomers and combinations thereof, are within the scope of the present invention.

The terms “R” and “S” are used herein as commonly used in organic chemistry to denote specific confirguation of a chiral center. The term “R” (rectus) refers to that configuration of a chiral center with a clockwise relationship of group priorities (highest to second lowest) when viewed along the bond toward the lowest priority group. The term “S” (sinister) refers to that configuration of a chiral center with a counterclockwise relationship of group priorities (highest to second lowest) when viewed along the bond toward the lowest priority group. The priority of groups is based upon their atomic number (in order of decreasing atomic number). A partial list of priorities and a discussion of stereochemistry is contained in Nomenclature of Organic Compounds: Principles and Practice, (J. H. Fletcher, et al., eds. 1974) at pages 103-120.

In addition to the (R)-(S) system, the older D-L system is also used in this document to denote absolute configuration, especially with reference to amino acids. In this system, a Fischer projection formula is oriented so that the number 1 carbon of the main chain is at the top. The prefix “D” is used to represent the absolute configuration of the isomer in which the functional (determining) group is on the right side of the carbon atom at the chiral center and “L”, that of the isomer in which it is on the left.

The term “pharmaceutically-acceptable salt” as used herein, refers to a salt of a compound of the above Formula (I). It should be recognized that the particular counterion forming a part of any salt of this invention is usually not of a critical nature, so long as the salt as a whole is pharmacologically acceptable and as long as the counterion does not contribute undesired qualities to the salt as a whole.

The compounds of Formula (I) described herein form pharmaceutically-acceptable acid addition salts with a wide variety of organic and inorganic acids and include the physiologically-acceptable salts which are often used in pharmaceutical chemistry. Such salts are also part of this invention. A pharmaceutically-acceptable acid addition salt is formed from a pharmaceutically-acceptable acid, as is well known in the art. Such salts include the pharmaceutically acceptable salts listed in Journal of Pharmaceutical Science, 66, 2-19 (1977), which are known to the skilled artisan. See also, The Handbook of Pharmaceutical Salts; Properties, Selection, and Use. P. H. Stahl and C. G. Wermuth (ED.s), Verlag, Zurich (Switzerland) 2002.

Typical inorganic acids used to form such salts include hydrochloric, hydrobromic, hydriodic, nitric, sulfuric, phosphoric, hypophosphoric, metaphosphoric, pyrophosphoric, and the like. Salts derived from organic acids, such as aliphatic mono and dicarboxylic acids, phenyl substituted alkanoic acids, hydroxyalkanoic and hydroxyalkandioic acids, aromatic acids, aliphatic and aromatic sulfonic acids, may also be used. Such pharmaceutically acceptable salts thus include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, α-hydroxybutyrate, butyne-1,4-dicarboxylate, hexyne-1,4-dicarboxylate, caprate, caprylate, cinnamate, citrate, formate, fumarate, glycollate, heptanoate, hippurate, lactate, malate, maleate, hydroxymaleate, malonate, mandelate, mesylate, nicotinate, isonicotinate, nitrate, oxalate, phthalate, teraphthalate, propiolate, propionate, phenylpropionate, salicylate, sebacate, succinate, suberate, benzenesulfonate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, p-toluenesulfonate, xylenesulfonate, tartarate, and the like.

Preferred compounds of this invention include compounds of formula I wherein R⁴ is CH₃. Further, preferred compounds include those wherein R¹ is C₆H₅CH₂OCH₂— or C₆H₅(CH₂)₃—. More preferred compounds are those where R³ is phenyl para-substituted by W. Further, more preferred compounds are those where W is F or —OCH₃. The skilled artisan will appreciate that additional preferred embodiments may be selected by combining the preferred embodiments above, or by reference to the examples given herein. Specific examples of compounds of formula (I) are as follows:

2-(2-Amino-2-methyl-propionylamino)-5-phenyl-pentanoic acid {1-[1-(4-methoxy-phenyl)-1-methyl-2-oxo-2-pyrrolidin-1-yl-ethyl]-1H-imidazol-4-yl}-amide;

2-(2-Amino-2-methyl-propionylamino)-5-phenyl-pentanoic acid {1-[1-(4-fluoro-phenyl)-1-methyl-2-oxo-2-pyrrolidin-1-yl-ethyl]-1H-imidazol-4-yl}-amide;

or pharmaceutically acceptable salts thereof.

The compounds of formula (I) are described in Kenneth Lee Hauser et al., U.S. Pat. No. 6,639,076 B1 and WO 00/49037, published 24 Aug. 2000, the disclosures of which are incorporated by reference herein as if fully set forth. The synthesis of the compounds of formula (I) are fully set forth as well as a disclosure that said compounds are useful as growth hormone secretagogues. As growth hormone secretagogues, the compounds have previously been disclosed as useful in the treatment of conditions associated with growth hormone deficiencies and age-related frailty, osteoporosis, and loss of muscle mass.

As used herein, the term “patient” refers to a warm-blooded animal or mammal which is in need of inhibiting nausea with or without emesis associated with migraine headache, head injury, “morning sickness” of pregnancy, chemotherapy, viral infections, cancer, hypoglycemia, vertigo, motion sickness, consumption of a noxious or toxic agent, and other paraphysiological conditions that result in sensation of nausea. It is understood that guinea pigs, dogs, cats, rats, mice, hamsters, and primates, including humans, are examples of patients within the scope of the meaning of the term. Preferred patients include humans.

As used herein, the term “treating” is defined to include its generally accepted meaning which includes preventing, prohibiting, restraining, and slowing, stopping or reversing progression, or severity, and holding in check and/or treating existing characteristics. The present method includes both medical therapeutic and/or prophylactic treatment, as appropriate.

As used herein, the term “therapeutically effective amount” means an amount of compound of the present invention which is capable of alleviating the symptoms of the various pathological conditions herein described. The specific dose of a compound administered according to this invention will, of course, be determined by the particular circumstances surrounding the case including, for example, the compound administered, the route of administration, the state of being of the patient, and the pathological condition being treated. A typical daily dose for human use will contain a nontoxic dosage level of from about 1 mg to about 1000 mg/day of a compound of the present invention. Preferred daily doses generally will be from about 10 mg to about 600 mg/day. Most preferred doses range may range from 20 mg to about 100 mg, administered once to three times per day.

A compound of formula (I) may be administered to a patient suffering from nausea, emesis or associated symptoms thereof. Once relief has been provided, the composition can be administered under a regimen to maintain a substantially symptom-free state. Generally, the dosage or frequency of administration of the composition of the invention to keep the patient essentially free of the complained of afflictions will be less than the dosage or frequency used in the initial phase of treatment. The dosage or frequency can be cut back until the ailments begin to manifest themselves once again. The dosage or frequency is then adjusted to just suppress the symptoms.

A compound of formula (I) can thus be provided as part of a chemotherapeutic regimen with the benefit that the patient is better able to withstand the discomfort associated with same. The compound can be administered one or more times daily as decided by the attending physician. Thereafter, the frequency of administration of the compound can be reduced to once a day or less for maintaining a symptom-free state.

The compounds of this invention can be administered by a variety of routes including rectal, oral, transdermal, subcutaneus, intravenous, intramuscular, and intranasal. These compounds preferably are formulated prior to administration, the selection of which will be decided by the attending physician. Thus, another aspect of the present invention is a pharmaceutical composition comprising an effective amount of a compound of Formula I, or a pharmaceutically acceptable salt thereof, and a pharmaceutically acceptable carrier, diluent, or excipient.

The total active ingredients in such formulations comprises from 0.1% to 99.9% by weight of the formulation. By “pharmaceutically acceptable” it is meant the carrier, diluent, excipients and salt must be compatible with the other ingredients of the formulation, and not deleterious to the recipient thereof.

Pharmaceutical formulations of the present invention can be prepared by procedures known in the art using well-known and readily available ingredients. For example, the compounds of formula I can be formulated with common excipients, diluents, or carriers, and formed into suppositories, tablets, capsules, suspensions, powders, and the like. Examples of excipients, diluents, and carriers that are suitable for such formulations include the following: fillers and extenders such as starch, sugars, mannitol, and silicic derivatives; binding agents such as carboxymethyl cellulose and other cellulose derivatives, alginates, gelatin, and polyvinyl-pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

The compounds also can be formulated as elixirs or solutions for convenient oral administration or as solutions appropriate for parenteral administration, for example, by intramuscular, subcutaneous or intravenous routes. Compounds of formula I, alone or in combination with a pharmaceutical agent of the present invention, generally will be administered in a convenient formulation.

The compounds of the present invention can be administered alone or in the form of a pharmaceutical composition, that is, combined with pharmaceutically acceptable carriers, or excipients, the proportion and nature of which are determined by the solubility and chemical properties of the compound selected, the chosen route of administration, and standard pharmaceutical practice. The compounds of the present invention, while effective themselves, may be formulated and administered in the form of their pharmaceutically acceptable salts, for purposes of stability, convenience of crystallization, increased solubility, and the like.

Thus, the present invention provides pharmaceutical compositions comprising a compound of the Formula (I) and a pharmaceutically acceptable diluent.

The compounds of Formula (I) can be administered by a variety of routes. In effecting treatment of a patient afflicted with or at risk of developing the disorders described herein, a compound of Formula (I) can be administered in any form or mode that makes the compound bioavailable in an effective amount, including oral and parenteral routes. For example, compounds of Formula (I) can be administered rectally, orally, by inhalation, or by the subcutaneous, intramuscular, intravenous, transdermal, intranasal, rectal, occular, topical, sublingual, buccal, or other routes. Oral administration may be preferred for treatment of the disorders described herein. However, oral administration is not the only preferred route because patients suffering with nausea have difficulty taking anything by mouth and if emesis occurs, may not absorb the complete dosage. Other routes include the intravenous route as a matter of convenience or to avoid potential complications related to oral administration. When the compound of Formula (I) is administered through the intravenous route, an intravenous bolus or slow infusion is preferred.

One skilled in the art of preparing formulations can readily select the proper form and mode of administration depending upon the particular characteristics of the compound selected, the disorder or condition to be treated, the stage of the disorder or condition, and other relevant circumstances. (Remington's Pharmaceutical Sciences, 18th Edition, Mack Publishing Co. (1990)).

The pharmaceutical compositions are prepared in a manner well known in the pharmaceutical art. The carrier or excipient may be a solid, semi-solid, or liquid material that can serve as a vehicle or medium for the active ingredient. Suitable carriers or excipients are well known in the art. The pharmaceutical composition may be adapted for oral, inhalation, parenteral, or topical use and may be administered to the patient in the form of tablets, capsules, aerosols, inhalants, suppositories, solutions, suspensions, or the like.

For the purpose of oral therapeutic administration, the compounds may be incorporated with excipients and used in the form of tablets, troches, capsules, elixirs, suspensions, syrups, wafers, chewing gums and the like. These preparations should contain at least 4% of the compound of the present invention, the active ingredient, but may be varied depending upon the particular form and may conveniently be between 4% to about 70% of the weight of the unit. The amount of the compound present in compositions is such that a suitable dosage will be obtained. Preferred compositions and preparations according to the present invention may be determined by a person skilled in the art.

The tablets, pills, capsules, troches, and the like may also contain one or more of the following adjuvants: binders such as povidone, hydroxypropyl cellulose, microcrystalline cellulose, gum tragacanth or gelatin; excipients such as dicalcium phosphate, starch, or lactose; disintegrating agents such as alginic acid, Primogel, corn starch and the like; lubricants such as talc, hydrogenated vegetable oil, magnesium stearate or Sterotex; glidants such as colloidal silicon dioxide; and sweetening agents, such as sucrose, aspartame, or saccharin, or a flavoring agent, such as peppermint, methyl salicylate or orange flavoring, may be added. When the dosage unit form is a capsule, it may contain, in addition to materials of the above type, a liquid carrier such as polyethylene glycol or a fatty oil. Other dosage unit forms may contain other various materials that modify the physical form of the dosage unit, for example, coatings. Thus, tablets or pills may be coated with sugar, shellac, or other coating agents. Syrups may contain, in addition to the present compounds, sucrose as a sweetening agent and certain preservatives, dyes and colorings and flavors. Materials used in preparing these various compositions should be pharmaceutically pure and non-toxic in the amounts used.

Since oral administration is often impossible, the composition is most preferably used in the form of a suppository, which is inserted into a patient's rectum, vagina, or otherwise administered across a patient's mucosal membrane. Also, the composition may be made available in a form suitable for parenteral administration, e.g., intravenous, intraperitoneal or intramuscular.

The composition of the invention is useful for providing relief to a patient experiencing an emetogenic condition. The present composition is particularly efficacious for treating patients undergoing, about to undergo, or recovering from chemotherapy for a deadly disease, such as cancer. However, other conditions, such as vertigo, motion sickness, AIDS, food poisoning and other acute or chronic diseases and infections that cause nausea, emesis, or associated symptoms thereof, may be effectively treated by the administration of the composition disclosed herein. In particular, the composition of the invention finds exceptional beneficial use in patients who have either exhausted all other medical alternatives or are considered terminally ill. In these patients (no matter what the cause of their illness) the composition provides exceptional relief of unwanted symptoms of nausea, vomiting and the like.

The compounds of Formula (I) are anti-emetic compounds. A preferred compound of formula (I) wherein R¹ is C₆H₅CH₂OCH₂—, Y is pyrrolidinyl; R³ is phenyl para-substituted by W; W is methoxy; and R⁴ is CH₃ may be prepared according to the methodologies disclosed in the above-referenced patents or according to Example 1 below and is referred to as “Compound 1” in Example 2. The anti-emetic activity of the compounds of Formula (I) may be demonstrated by the method described in Example 2.

General Comments.

All chemical syntheses may be carried out under a nitrogen atmosphere where appropriate. All procedures may utilize anhydrous solvents where appropriate. Melting points may be determined in open glass capillaries by use of a Thomas-Hoover apparatus, and are uncorrected. The ¹H NMR spectra are recorded at 300 MHz with a Bruker ARX 300 spectrometer. Electrospray mass spectral analysis is obtained on a Micromass ZQ. Analysis (tlc) is performed on pre-coated glass plates (0.25 mm) with Silica Gel 60F₂₅₄ (E. Merck, Darmstad). Flash chromatography is performed with Silica Gel 60 (230-400 mesh, E. Merck, Darmstad). All solvents and reagents may be purchased from Sigma-Aldrich Corporation.

### EXAMPLE 1

### Preparation of 2-(2-Amino-2-methyl-propionylamino)-5-phenyl-pentanoic acid {1-[1-(4-methoxy-phenyl)-1-methyl-2-oxo-2-pyrrolidin-1-yl-ethyl]-1H-imidazol-4-yl}-amide

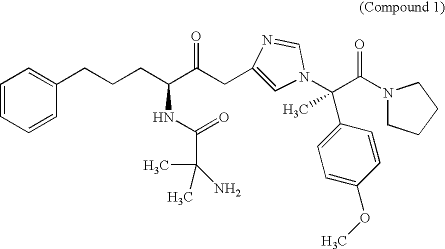

(a) Methoxy-phenyl)-(4-nitro-imidazol-1-yl)-acetic acid ethyl ester (8). To a solution of a compound of the formula 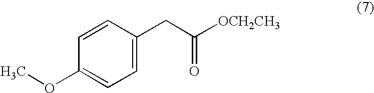
(40 g, 200 mmol) in carbon tetrachloride (500 mL) is added N-bromosuccinimide (37 g, 206 mmol) and 4 drops of 48% HBr. The reaction mixture is refluxed for 5 h, filtered and concentrated to dryness. The resulting oil is flash chromatographed on silica gel using chloroform as eluant to afford 49.5 g (91%) of the bromide as a colorless oil. This material is immediately dissolved in DMF (500 mL) and to this is added 4-nitroimidazole (20.5 g, 181 mmol) and potassium carbonate (75 g, 543 mmol). The reaction mixture is stirred overnight at ambient temperature, filtered and concentrated to dryness. The resulting oil is partitioned between ethyl acetate and water and extracted with ethyl acetate. The combined organics are washed with brine, dried over sodium sulfate, filtered and concentrated to dryness. The resulting oil is absorbed onto a silica pad and flash chromatographed on silica gel using 30-70% ethyl acetates/hexanes to yield 8 (33.6 g, 61%) as an orange oil that solidifies upon sitting. ¹H-NMR (300 MHz, DMSO): 1.17 (t, J=7.2 Hz, 3H), 3.78 (s, 3H), 4.25 (q, J=7.2 Hz, 2H), 6.57 (s, 1H), 7.02 2H), 7.46 (d, J=8.7 Hz, 2H), 7.92 (s, 1H), 8.38 (s, 1H); Anal. Calc'd for C₁₄H₁₅B₃O₅: C, 55.08; H, 4.95; N, 13.76. Found: C, 54.93; H, 4.89; N, 13.82; MS m/z 306 (M⁺).

(b) 2-(4-Methoxy-phenyl)-2-(4-nitro-imidazol-1-yl)-propionic acid ethyl ester (9). A solution of 8 (0.0710 moles, 21.68 g) in THF (210 mL) is cooled to 0° C. Sodium bis(trimethylsilyl)amide (1.0M in THF, 75 ml, 0.07455 moles) is added dropwise over 1 hour maintaining 0-5° C. The mixture was stirred for 30 minutes at 0° C. Methyl iodide (5.0 ml, 0.08023 moles) is added dropwise over 30 minutes maintaining 0-5° C. The reaction is stirred 1.5 hours at 0° C. until complete as monitored by TLC. The reaction mixture is concentrated on the rotary to 150 ml. The concentrated residue is partitioned between ethyl acetate (200 ml) and water (200 ml). The layers were separated. The aqueous layer is extracted with ethyl acetate (2×100 ml), and the combined organic layers were washed with brine (2×100 ml). The organic layer is dried with sodium sulfate and then filtered. The filtrate is concentrated on a rotary to give 9 as a colorless oil (20.95 g, 92% yield). ¹H NMR (300 MHz, CDCl₃): 1.21 (t, J=6.2 Hz, 3H), 2.19 (s, 3H), 3.83 (s, 3H), 4.23-4.27 (m, 2H), 6.95 (d, J=7.8 Hz, 2H), 7.17 (d, J=7.8 Hz, 2H), 7.32 (d, J=1.8 Hz, 1H), 7.65 (d, J=1.8 Hz, 1H); Anal. calcd. for C₁₅H₁₇N₃O₅; 56.42 C, 5.37 H, 13.16 N; found 56.13 C, 5.35 H, 13.01 N; MS m/z 320 (M⁺).

(c) 3-[2-(4-Methoxy-phenyl)-2-(4-nitro-imidazol-1-yl)-propionyl]-4-methyl-5-phenyl-oxazolidin-2-one (10, 11). A solution of 9 (8.35 g, 28.89 mmol) in THF (100 mL) is treated with lithium hydroxide (1.82 g, 43.34 mmol) and water (50 mL). The reaction is stirred at ambient temperature for 30 minutes. Water is added and the mixture washed with diethyl ether. The pH of the aqueous layer is adjusted to 3.0 with 10% sodium bisulfate. The mixture is saturated with sodium chloride and washed with ethyl acetate. The ethyl acetate washes are combined, dried over sodium sulfate, filtered, and concentrated in vacuo. The resulting crude solid is dissolved in anhydrous dichloromethane (100 mL) under nitrogen. To this solution is added catalytic DMF (0.1 mL) and excess oxalyl chloride (25 g). This mixture is stirred 3 hours, then concentrated in vacuo. The resulting crude foam is dissolved in THF (20 mL) and added dropwise to a solution of lithium (4R, 5S)-(+)-4-methyl-5-phenyl-2-oxazolidinone [generated by adding n-BuLi (1.6M in hexanes, 19.9 mL, 31.82 mmol) dropwise to a solution of(4R, 5S)-(+)-4-methyl-5-phenyl-2-oxazolidinone (5.64 g, 31.82 mmol) in THF (50 mL) at −78° C. under nitrogen. This solution is stirred 20 min., then used without further purification.]. The resulting mixture is stirred at −78° C. for 30 min., then warmed to 0 C. The mixture is quenched with saturated sodium bicarbonate. Ethyl acetate and water are added and the mixture washed with sodium bicarbonate and brine. The organic layer is dried over sodium sulfate, filtered, and concentrated in vacuo. The resulting foam is purified by flash chromatography (400 g silica, 5% diethyl ether/dichloromethane) to yield 10 (2.79 g, 41% yield) and 11 (2.80 g, 41%) of the desired product as colorless foams: 10—¹H NMR (300 MHz, CDCl₃): 0.95 (d, J=6.8 Hz, 3H), 2.52 (s, 3H), 3.85 (s, 3H), 4.81-4.92 (m, 1H), 7.76 (d, J=7.4 Hz, 1H), 6.98 (d, J=8.6 Hz, 2H), 7.12 (d, J=1.7, 1H), 7.22-7.28 (m, 5H), 7.38-7.40 (m, 3H), 7.53 (d, J=1.7 Hz, 1 Hz); Anal. calcd. for C₂₃H₂₂N₄O₆; 61.33 C, 4.92 H, 12.44 N; found 60.92 C, 4.82 H, 12.03 N; MS mz/z 451 (M⁺): 11—¹H NMR (300 MHz, CDCl₃): 0.97 (d, J=6.3 Hz, 3H), 2.50 (s, 3H), 3.85 (s, 3H), 4.80-4.91 (m, 1H), 5.73 (d, J=7.4 Hz, 1H), 6.97 (d, J=8.6 Hz, 2H), 7.06 (d, J=1.7 Hz, 1H), 7.19-7.22 (m, 4H), 7.33-7.35 (m, 3H), 7.51 (d, J=1.7H); Anal. calcd. for C₂₃H₂₂N₄O₆; 61.33 C, 4.92 H, 12.44 N; found 61.57 C, 4.98 H, 12.47 N; MS m/z 451 (M⁺).

(d) 2-(4-Methoxy-phenyl)-2-(4-nitro-imidazol- 1 -yl)- 1 -pyrrolidin-1-yl-propan-1-one (12). A solution of 11 (1.25 g, 2.78 mmol) in THF (50 mL) is added to a solution of lithium hydroxide (0.14 g, 3.33 mmol) in water (25 mL). The resulting mixture is stirred at ambient temperature for 30 minutes. Water is added and the mixture washed with diethyl ether. The pH of the aqueous layer is adjusted to 3.0 with 10% aqueous sodium bisulfate. The mixture is saturated with sodium chloride and washed with ethyl acetate. The ethyl acetate washes are combined, dried over sodium sulfate, filtered, and concentrated in vacuo. The resulting crude solid is dissolved in anhydrous dichloromethane (50 mL) under nitrogen. To this solution is added catalytic DMF (0.1 mL) and excess oxalyl chloride (5 g). This mixture is stirred 3 hours, then concentrated in vacuo. The resulting crude foam is dissolved in anhydrous dichloromethane (50 mL) and cooled to 0 C. 4-Dimethylaminopyridine (catalytic, 10 mg) and pyrrolidine (0.24 mL, 2.89 mmol) are added and the resulting solution stirred for 18 hours. Dichloromethane is then added and the mixture washed with sodium bicarbonate and brine. The organic layer is dried over sodium sulfate, filtered, and concentrated in vacuo. The crude foam was purified by flash chromatography (silica, 100 g, 5% methanol/dichloromethane) to yield 12 (0.78 g, 86% yield) as a colorless foam: ¹H NMR (300 MHz, CDCl₃): 1.60-1.62 (m, 1H), 1.79-1.83 (m, 3H), 2.22 (s, 3H), 2.65-2.68 (m, 1H), 3.02-3.05 (m, 1H), 3.61-3.66 (m, 2H), 3.95 (s, 3H), 7.00 (d, J=8.5 Hz, 2H), 7.21 (d, J=1.7 Hz, 1H), 7.25 (d, J=8.5, 2H), 7.55 (d, J=1.7 Hz, 1H); Anal. calcd. for C₁₇H₂₀N₄O₄; 59.59 C, 5.85 H, 16.27 N; found 59.59 C, 5.96 H, 16.19 N; MS m/z 345 (M⁺).

(e) [1-(1-{1-[1-(4-Methoxy-phenyl)-1-methyl-2-oxo-2-pyrrolidin-1-yl-ethyl]-1H-imidazol-4-ylcarbamoyl}-4-phenyl-butylcarbamoyl)-1-methyl-ethyl]-carbamic acid tert-butyl ester (13). A solution of 12 (0.77 g, 2.24 mmmol) in THF (5 mL) is added to a suspension of 5% palladium on carbon (0.80 g, catalytic, 25 mL THF) under inert atmosphere. The resulting mixture is placed under hydrogen (40 psi) on a Parr shaker for 1.5 hours. The resulting mixture is placed under nitrogen and celite added. The mixture is then filtered and rinsed with THF. The filtrate is place under nitrogen and HOBT (0.30 g, 2.46 mmol), 2-(2-tert-butoxycarbonylamino-2-methyl-propionylamino)-5-phenyl-pentanoic acid (2.01 g, 5.29 mmol), and DCC (0.51 g, 2.46 mmol) were added. The resulting mixture is stirred 18 hours at ambient temperature then concentrated in vacuo. The crude material is dissolved in ethyl acetate and washed with sodium bicarbonate and brine. The organic layer is dried over sodium sulfate, filtered, and concentrated in vacuo. The resulting crude foam is purified by flash chromatography (silica, 50 g, 2% methanol/dichloromethane) to yield 13 (0.70 g, 46% yield) as a light yellow foam: ¹H NMR (300 MHz, CDCl₃): 1.40 (s, 9H), 1.51 (s, 6H), 1.65-1.80 (m, 7H), 2.00-2.03 (m, 1H), 2.15 (s, 3H), 2.20-2.22 (m, 1H), 2.40-2.45 (m, 1H), 2.58-2.63 (m, 2H), 2.93-2.95 (m, 2H), 3.60-3.63 (m, 2H), 3.85 (s, 3H), 4.58-4.62 (m, 1H), 5.03 (s, 1H), 6.83 (d, J=7.8 Hz, 1H), 6.95 (d, J=7.8 Hz, 2H), 7.10-7.23 (m, 7H), 7.31 (d, J=1.7 Hz, 1H), 9.75 (bs, 1H); Anal. calcd. for C₃₇H₅₀N₆O₆; 65.85 C, 7.47 H, 12.45 N; found 65.83 C, 7.27 H, 12.38 N; MS m/z 675 (M⁺).

(f) 2-(2-Amino-2-methyl-propionylamino)-5-phenyl-pentanoic acid {1-[1-(4-methoxy-phenyl)-1-methyl-2-oxo-2-2-pyrrolidin-1-yl-ethyl]-1H-imidazol-4-yl}-amide (Compound 1). A solution of 13 (1.01 grams, 0.0015 mol) dissolved in methylene chloride (7 mL) is treated with a 2.25M solution of anhydrous hydrochloric acid in ethyl acetate. The reaction is stirred at ambient temperature until complete as determined by TLC (2 hours). The reaction mixture is evaporated to dryness and the residue partitioned between a saturated sodium bicarbonate solution (50 mL) and ethyl acetate (3×50 mL). The ethyl acetate extracts are combined, dried using sodium sulfate, and evaporated to dryness to give 0.77 grams (90%) of crude Compound 1. This material is recrystalized from ethanol to yield Compound 1 (0.58 g, 67%) as a white solid; mp 197-198° C. ¹H NMR (500 MHz, DMSO-d₆): δ 1.12 (s, 3H), 1.14 (s, 3H), 1.45-1.6 (m, 4H), 1.61-1.65 (m, 4H), 1.96 (bs, 2H), 2.08 (s, 3H), 2.50-2.56 (m, 3H), 2.90-2.94 (m, 1H), 3.38-3.42 (m, 2H), 3.76 (s, 3H), 4.18 (bs, 1H), 6.87 (d, J=1.3, 1H), 6.99 (d, J=8.8 Hz, 2H), 7.10-7.13 (m, 3H), 7.20-7.24 (m, 5H), 8.07 (bs, 1H). MS m/z 575.3 (M⁺); Anal. Calcd. For C₃₂H₄₂N₆O₄: C, 66.88; H, 7.37; N, 14.62. Found: C, 66.60; H, 7.19; N, 14.45.

### EXAMPLE 2

### Preparation of 2-(2-Amino-2-methyl-propionylamino)-5-phenyl-pentanoic acid {1-[1-(4-fluoro-phenyl)-1-methyl-2-oxo-2-pyrrolindin-1-yl-ethyl]-1H-imidazol-4-yl}-amide

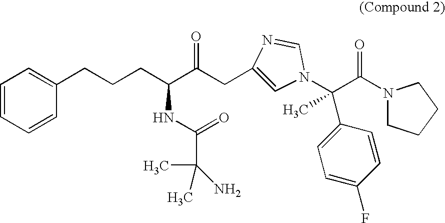

(a) Ethyl 4-fluorophenyl acetate. To a solution of p-fluorophenylacetic acid (50 g, 324 mmol)in absolute EtOH (300 mL) add catalytic p-toluene sulfonic acid (7 g) and heat the resulting mixture to reflux for 30 min. Concentrate the reaction in vacuo and purify by flash chromatography (100% chloroform) to yield the desired product (59 g, 100%) as a clear oil. ¹H NMR (300 MHz, CDCl₃)—consistent with structure; FDMS (M+) 182.

(b) (4-Fluoro-phenyl)-(4-nitro-imidazol-1-yl)-acetic acid ethyl ester. To a solution of ethyl p-fluorophenyl acetate (61.0 g, 333 mmol) in carbon tetrachloride (300 mL) is added N-bromosuccinamide (61 g, 343 mmol) and HBr (4 drops, 48%) and the resulting mixture is refluxed for 3 hours. Cool the reaction to ambient temperature, filter, and concentrate the filtrate in vacuo to yield the crude α-bromo ester (68 g, 78%). A solution of the crude α-bromo ester (68.00 g, 260.0 mmol) in DMF (300 mL) is treated with 4-nitroimidazole (35.0 g, 312 mmol) and potassium carbonate (108.0 g, 780.0 mmol). Stir the mixture 18 hours at ambient temperature. Filter the mixture and concentrate the filtrate in vacuo. Dissolve the resulting crude material in ethyl acetate and extract with saturated sodium bicarbonate followed by brine. Dry the organic layer over sodium sulfate, filter, and concentrate in vacuo. The resulting foam is purified by flash chromatography (400 g silica, ethyl acetate/hexanes gradient) to yield the desired product (39.8 g, 52%) as a light orange oil. ¹H NMR (300 MHz, CDCl₃)—consistent with structure; Anal. calcd. for C₁₃H₁₂FN₃O₄; 53.24 C, 4.12 H, 14.33 N; found 53.51 C, 4.07 H, 14.43 N; FDMS (M+) 294.

(c) 2-(4-Fluoro-phenyl)-2-(4-nitro-imidazol-1-yl)-propionic acid ethyl ester. A solution of the compounds of Example 2, step b (27.19 g, 92.80 mmol) in THF (200 mL) is added dropwise to a solution of sodium bis(trimethylsilyl) amide (1.0 M in THF, 102.0 mL, 102.0 mmol) under nitrogen at 0° C. Stir the mixture for 10 min., then add methyl iodide (1.1 mL, 18.03 mmol) dropwise. Stir the reaction thirty minutes at 0° C., then 1 h at ambient temperature. Quench the mixture with a saturated solution of sodium bicarbonate. Add ethyl acetate and wash the mixture with bicarbonate followed by brine. Dry the organic layer over sodium sulfate, filter, and concentrate in vacuo. Purify the resulting foam by flash chromatography (200 g silica, 1:1 ethyl acetate/hexanes) to yield the desired product (26.93 g, 95%) as a light orange oil. ¹H NMR (300 MHz, CDCl₃)—consistent with structure; FIMS (M+) 308.

(d) 3-[2-(Fluoro-phenyl)-2-(4-nitro-imidazol-1-yl)-propionyl]-4-methyl-5-phenyl-oxazolidin-2-one. Treat a solution of a compound of Example 2, step c (26.93 g, 87.71 mmol) in THF (150 mL) with lithium hydroxide (4.42 g, 105.26 mmol) and water (75 mL). Stir the reaction at ambient temperature for 30 minutes. Add water and wash the mixture with diethyl ether. Adjust the pH of the aqueous layer to 3.0 with 10% sodium bisulfate. Saturate the mixture with sodium chloride and wash with ethyl acetate. Combine the ethyl acetate washes, dry over sodium sulfate, filter, and concentrate in vacuo. Dissolve the resulting crude solid in anhydrous dichloromethane (200 mL) under nitrogen. To this solution is added catalytic DMF (0.5 mL) and oxalyl chloride (22.1 mL, 253.65 mmol). Stir this mixture for 3 hours, then concentrate in vacuo. Dissolve the resulting crude foam in THF (20 mL) and add dropwise to a solution of lithium (4R, 5S)-(+)-4-methyl-5-phenyl-2-oxazolidinone [generated by adding n-BuLi (1.6M in hexanes, 79.2 mL, 126.82 mmol) dropwise to a solution of (4R, 5S)-(+)-4-methyl-5-phenyl-2-oxazolidinone (22.40 g, 126.82 mmol) in THF (200 mL) at −78° C. under nitrogen. This solution is stirred 20 min., then used without further purification]. Stir the resulting mixture at −78° C. for 30 min., then warm to 0 C. Quench the mixture with saturated sodium bicarbonate. Add ethyl acetate and water and wash the mixture with sodium bicarbonate and brine. Dry the organic layer over sodium sulfate, filter, and concentrate in vacuo. Purify the resulting foam by flash chromatography (400 g silica, 5% diethyl ether/dichloromethane) to yield diastereomer 1 (16.66 g, 45% yield) and diastereomer 2 (13.32 g, 36%) of the desired product as colorless foams. ¹H NMR (300 MHz, CDCl₃)—consistent with structure; FIMS (M+) 439.

(e) 2-(4-Fluoro-phenyl)-2-(4-nitro-imidazol- 1 -yl) 1-pyrrolidin-1-yl-propan-1-one. Add a solution of a compound of Example 2, step d (11.00 g, 25.23 mmol) in THF (100 mL) to a solution of lithium hydroxide (1.16 g, 27.75 mmol, 50 mL water). Stir the resulting mixture at ambient temperature for 30 minutes. Add water and wash the mixture with diethyl ether. Adjust the pH of the aqueous layer to 3.0 with 10% aqueous sodium bisulfate. Saturate the mixture with sodium chloride and wash with ethyl acetate. Combine the ethyl acetate washes, dry over sodium sulfate, filter, and concentrate in vacuo. Dissolve the resulting crude acid (2.00 g, 7.17 mmol) in anhydrous dichloromethane (50 mL) under nitrogen. To this solution is added catalytic DMF (0.1 mL) and excess oxalyl chloride (5 g). Stir this mixture for 3 hours, then concentrate in vacuo. Dissolve the resulting crude foam in anhydrous dichloromethane (50 mL) and cool to 0 C. 4-Dimethylaminopyridine (catalytic, 10 mg) and pyrrolidine (1.8 mL, 21.51 mmol) are added and the resulting solution is stirred for 18 hours. Add dichloromethane and wash the mixture with sodium bicarbonate and brine. Dry the organic layer over sodium sulfate, filter, and concentrate in vacuo. Purify the crude foam by flash chromatography (silica, 100 g, 3% methanol/dichloromethane) to yield the desired product (1.84 g, 77% yield) as a colorless foam. ¹H NMR (300 MHz, CDCl₃)—consistent with structure; Anal. calcd. for C₁₆H₁₇FN₄O₃; 57.83 C, 5.16 H, 16.86 N; found 57.85 C, 5.01 H, 16.48 N; FIMS (M+) 333.

(f) [1-(1-{1-[1-(4-Fluoro-phenyl)1-methyl-2-oxo-2-pyrrolidin-1-yl-ethyl]-1H-imidazol-4-ylcarbamoyl}-4-phenyl-butylcarbamoyl)-1-methyl-ethyl]-carbamic acid tert-butyl ester. Add a solution of a compound of Example 2, step e (1.84 g, 5.54 mmmol) in THF (10 mL) to a suspension of 5% palladium on carbon (1.95 g, catalytic, 40 mL THF) under inert atmosphere. Place the resulting mixture under hydrogen (40 psi) on a Parr shaker for 1.5 hours. Place the resulting mixture under nitrogen and add celite. Filter the mixture, rinse with THF, and split the resulting solution in half. Place the filtrate under nitrogen and add HOBT (0.37 g, 2.77 mmol), a compound of the formula 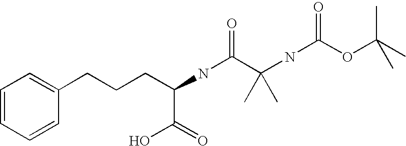
(ref. Moriello et al., U.S. Pat. No. 5,492,916, issued Feb. 20, 1996) (1.05 g, 2.77 mmol), and DCC (0.63 g, 3.05 mmol). Stir the resulting mixture 18 hours at ambient temperature, then concentrate in vacuo. Dissolve the crude material in ethyl acetate and wash with sodium bicarbonate and brine. Dry the organic layer over sodium sulfate, filter, and concentrate in vacuo. Purify the resulting crude foam by flash chromatography (silica, 100 g, 3% methanol/dichloromethane) to yield the desired product (1.17 g, 64% yield) as a light yellow foam. ¹H NMR (300 MHz, CDCl₃)—consistent with structure; FIMS (M+) 663, (M−) 661.

(g) 2-(2-Amino-2-methyl-propionylamino)-5-phenyl-pentanoic acid {1-[1-(4-fluoro-phenyl)-1-methyl-2-oxo-2-pyrrolindin-1-yl-ethyl]-1H-imidazol-4-yl}-amide. Stir a solution of a compound of Example 2, step f (1.12 g, 1.69 mmol) in dichloromethane (20 mL) under nitrogen with anisole (0.2 mL) and triflouroacetic acid (5.0 mL) at ambient temperature for 3 hours. Quench the mixture with saturated sodium bicarbonate. Add ethyl acetate and stir the mixture 10 min. at ambient temperature. Wash the mixture with bicarbonate and brine, dry over sodium sulfate, filter, and concentrate in vacuo. Dissolve the crude material in ethyl acetate (10 mL) and add hydrochloric acid (saturated) in diethyl ether (5 mL). Filter the mixture to yield 0.86 g of the desired product as the crude HCl salt. Suspend this material in ethyl acetate and stir with saturated aq. sodium bicarbonate for one hour. Extract the organic layer with saturated aq. sodium bicarbonate, brine, dried over sodium sulfate, and concentrate in vacuo to a crude oil which after flash chromatographty (7% methanou/dichloromethane) yields the desired product (0.48 g, 50%) as a light yellow foam. ¹H NMR (300 MHz, CDCl₃)—consistent with structure; FIMS (M+) 563, (M−) 561.

### EXAMPLE 3

Procedure: On Day-1, male Long-Evans rats (n=6-8/group) are Vehicle dosed (1 ml/kg po) 30 minutes prior to the onset of the dark cycle. On the experimental day, rats are separated into four groups and dosed accordingly: Veh/Veh, Compound 1/Veh, Compound 1/Ipecac, or Veh/Ipecac. The timing of the ipecac dosing relative to the Veh or Compound 1 or 2 (30 mg/kg) dosing is varied (30 min or 2 hr), since the ability of Compound 1 or 2 to counter the effect of ipecac may be time dependent. Below is the 24-hr food intake and 24-hr change in body weight. The time between dosing of the Compound 1 and the ipecac is noted in the parentheses. In this example, Compound 1 corresponds to the compound of EXAMPLE 1 above and Compound 2 corresponds to the compound of EXAMPLE 2 above.

TABLE

| 24-hr | | |
|---|---|---|
| Food Intake | (30 min) | (2-hr) |
| Veh/Veh | 27.0 ± 1.5 g | 26.9 ± 2.0 g |
| Cmpd 1/Veh | 29.3 ± 1.2 g | 29.7 ± 1.1 g |
| Cmpd 1/Ipecac | 27.0 ± 0.8 g | 27.9 ± 0.8 g |
| Veh/Ipecac | 20.4 ± 2.3 g* | 21.6 ± 1.0 g* |
| Change in | | |
| Body Weight | (30 min) | (2-hr) |
| Veh/Veh | 4.7 ± 2.0 g | 0.1 + 1.7 g |
| Cmpd 1/Veh | 7.6 ± 1.4 g | 6.5 + 1.8 g |
| Cmpd 1/Ipecac | 6.4 ± 1.2 g | 7.1 + 1.7 g |
| Veh/Ipecac | −2.4 ± 5.4 g | −5.8 + 3.3 g* |
| *= p < 0.05 vs. all other groups | | |
| 24-hr | | |
| Food Intake | (30 min) | (2-hr) |
| Veh/Veh | 25.5 ± 1.0 g | 25.6 ± 1.0 g |
| Cmpd 2/Veh | 30.7 ± 1.1 g* | 27.8 ± 1.2 g* |
| Cmpd 2/Ipecac | 29.3 ± 1.6 g | 25.7 ± 1.1 g* |
| Veh/Ipecac | 22.3 ± 3.1 g | 18.9 ± 2.6 g |
| Change in | | |
| Body Weight | (30 min) | (2-hr) |
| Veh/Veh | 1.9 ± 1.3 g | 0.1 + 1.6 g |
| Cmpd 2/Veh | 10.2 ± 1.5 g* | 4.4 + 1.2 g# |
| Cmpd 2/Ipecac | 9.6 ± 1.2 g* | 3.4 + 1.8 g |
| Veh/Ipecac | −4.0 ± 5.4 g | −6.1 + 4.4 g |

*= p < 0.05 vs. Veh/Ipecac
#= p = 0.052 vs. Veh/Ipecac

Ipecac provided the nauseating stimulus to reduced food intake and boy weight in the rats. Both Compound 1 and Compound 2 (30 mg/kg) were able to stimulate food intake and body weight gain with the ipecac challenge (30 min or 2-hr).

1. A method comprising administering to a patient in need of treatment for nausea, emesis, or symptoms associated therewith a compound of formula (I) 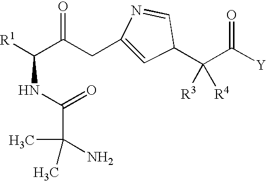
wherein:
R¹ is C₆H₅CH₂OCH₂—, C₆H₅(CH₂)₃— or indol-3-ylmethyl;
Y is pyrrolidinyl, 4-methyl-piperidinyl or NR²R²;
R² are each independently C₁-C₆ alkyl;
R³ is 2-napthyl or phenyl para-substituted by W;
W is H, F, CF₃, C₁-C₆ alkoxy or phenyl; and
R⁴ is H or methyl;
or a pharmaceutically acceptable salt or solvate thereof, in an amount that is effective in treating nausea, emesis, or symptoms associated therewith in said patient.

2. A method according to claim 1 wherein R⁴ is CH₃.

3. A method according to either of claims 1 or 2 wherein R³ is phenyl para-substituted by W.

4. A method according to claim 3 wherein W is F or methoxy.

5. A method according to claim 3 wherein Y is pyrrolidinyl.

6. A method according to claim 1 wherein said compound is 2-(2-amino-2-methyl-propionylamino)-5-phenyl-pentanoic acid {1-[1-(4-methoxy-phenyl)-1-methyl-2-oxo-2-pyrrolidin-1-yl-ethyl]-1H-imidazol-4-yl}-amide; or a pharmaceutically acceptable salt thereof.

7. A method according to claim 1 wherein said compound is 2-(2-amino-2-methyl-propionylamino)-5-phenyl-pentanoic acid {1-[1-(4-fluoro-phenyl)-1-methyl-2-oxo-2-pyrrolidin-1-yl-ethyl]-1H-imidazol-4-yl}-amide; or a pharmaceutically acceptable salt thereof.

8. A method according to claim 1 wherein said patient is a human.

9. A method according to claim 1 wherein said patient is diagnosed as suffering from an emetogenic condition.

10. A method according to claim 1 wherein said patient is undergoing chemotherapy, about to undergo chemotherapy, or is recovering from chemotherapy.

11. A method according to claim 1 wherein the patient is terminally ill.

12. A method according to claim 11 in which the patient's metabolic functions have failed.
